(19) United States
(12) Patent Application Publication
(10) Pub. No.: US 2020/0222302 A1
(43) Pub. Date: Jul. 16, 2020

(54) FILM FORMING COMPOSITIONS HAVING A TACK-FREE SURFACE

(71) Applicant: Dow Silicones Corporation, Midland, MI (US)

(72) Inventors: Thomas D. Bekemeier, Birch Run, MI (US); Patrick J. Fryfogle, Sanford, MI (US); Gary M. Wieber, Midland, MI (US); Kimmai T. Nguyen, Midland, MI (US); Zhi Li, Audubon, PA (US); Julie Cook, Turner, MI (US); Jason Vogel, Freeland, MI (US)

(21) Appl. No.: 16/624,160

(22) PCT Filed: Jun. 15, 2018
(86) PCT No.: PCT/US2018/037684

§ 371 Date: Dec. 18, 2019

Related U.S. Application Data
(60) Provisional application No. 62/552,482, filed on Aug. 31, 2017.

(51) Int. Cl. A61K 8/894; C09D 183/06; A61Q 1/00

(52) U.S. Cl. CPC A61K 8/894; C08G 77/12; A61Q 1/00; C09D 183/06

(57) ABSTRACT

This invention relates to a film forming composition that can produce a tack free surface comprising: (I) a hydrosilylation reaction product of: (a) a silicone resin having the formula: (R¹R²₂SiO_{1/2})_{w}(R¹R²SiO_{2/2})ₓ(R²SiO_{3/2})_{y}(SiO_{4/2})_{z} wherein R² is C₁ to C₁₀ hydrocarbon group free of aliphatic unsaturation, R¹ is R² or an alkenyl group, w is from 0.3 to 0.6, x is from 0.0 to 0.2, y is 0, z is from 0.4 to 0.7, w+x+y+z=1.0, and w+z is from 0.85 to 1.0, with the proviso that the silicone resin (a) has at least two silicon-bonded alkenyl groups per number average molecule; (b) a polyether compound having the formula: R³O(C₂H₄))_{c}(C₃H₆O)_{d}R³ wherein R³ is a monovalent unsaturated aliphatic hydrocarbon group containing 2 to 12 carbon atoms, c is from 0 to 50, d is from 0 to 50, and the value of c+d is greater than zero; (c) an SiH functional organopolysiloxane having the general formula: H_{c}R_{3-c}SiO(R₂SiO)ₐ(RHSiO)_{b}SiR_{3-c}H_{c} wherein c is 0 or 1, R can be identical or different and is a monovalent, optionally substituted hydrocarbon group with 1 to 18 carbon atoms per molecule, a and b are integers, with the proviso that the sum of a+b is 0 to 300, and wherein the organopolysiloxane contains SiH in amounts from 0.01 to 0.4 wt %; (d) a hydrosilylation catalyst; and (e) a carrier solvent; and (II) a silicone resin having the formula: (R⁴₃SiO_{1/2})ᵤ(SiO_{4/2})ᵥ wherein R⁴ is a C₁ to C₁₀ hydrocarbon group free of aliphatic unsaturation, u is from 0.3 to 0.6, v is from 0.4 to 0.7, and the value of u+v is 1.0. The film forming composition of this invention are useful in manufacturing personal care compositions, which may also be described as a personal care product composition.

### CROSS-REFERENCE TO RELATED APPLICATIONS

This application claims the benefit of U.S. Provisional Patent Application Ser. No. 62/552482 filed on 31 Aug. 2017 under 35 U.S.C. § 119 (e). U.S. Provisional Patent Application Ser. No. 62/552482 is hereby incorporated by reference

### BACKGROUND OF THE INVENTION

This invention relates to a film forming composition that can form a tack free film on the surface of a substrate. In personal care, and particularly in beauty care, the ability of a film forming composition to be tack free is very advantageous. In general, silicone film forming compositions are known in the art, with the most widely used silicone film former being MQ resin. MQ resins form a continuous film but are inflexible. Silicone-based alternatives to MQ resins are often more flexible but impart to the film a tacky feel on the skin. This invention offers flexibility to beauty care formulations without a imparting a tacky feel. Additionally, this invention imparts improved sebum resistance compared to benchmark formulations.

### BRIEF SUMMARY OF THE INVENTION

This invention relates to a film forming composition that can produce a tack free surface comprising: (I) a hydrosilylation reaction product of: (a) a silicone resin having the formula: (R¹R²₂SiO_{1/2})_{w}(R¹R²SiO_{2/2})ₓ(R²SiO_{3/2})_{y}(SiO_{4/2})_{z} wherein R² is a C₁ to C₁₀ hydrocarbon group free of aliphatic unsaturation, R¹ is R² or an alkenyl group, w is from 0.3 to 0.6, x is from 0.0 to 0.2, y is 0, z is from 0.4 to 0.7, w+x+y+z=1.0, and w+z is from 0.85 to 1.0, with the proviso that the silicone resin (a) has at least two silicon-bonded alkenyl groups per number average molecule; (b) a polyether compound having the formula: R³O(C₂H₄O)_{c}(C₃H₆O)_{d}R³ wherein R³ is a monovalent unsaturated aliphatic hydrocarbon group containing 2 to 12 carbon atoms, c is from 0 to 50, d is from 0 to 50, and the value of c+d is greater than zero; (c) an SiH functional organopolysiloxane having the general formula: H_{c}R_{3-c}SiO(R₂SiO)ₐ(RHSiO)_{b}SiR_{3-c}H_{c} wherein c is 0 or 1, R can be identical or different and is a monovalent, optionally substituted hydrocarbon group with 1 to 18 carbon atoms per molecule, a and b are integers, with the proviso that the sum of a+b is 0 to 300, and wherein the organopolysiloxane contains SiH in amounts from 0.01 to 0.4 wt %; (d) a hydrosilylation catalyst; and (e) a carrier solvent; and (II) a silicone resin having the formula: (R⁴₃SiO_{1/2})ᵤ(SiO_{4/2})ᵥ wherein R⁴ is a C₁ to C₁₀ hydrocarbon group free of aliphatic unsaturation, u is from 0.3 to 0.6, v is from 0.4 to 0.7, and the value of u+v is 1.0.

### DETAILED DESCRPTION OF THE INVENTION

In the hydrosilylation reaction product, component (a), the silicone resin, the alkenyl groups represented by R¹, which may be the same or different, typically have from 2 to about 10 carbon atoms, alternatively from 2 to 6 carbon atoms, and are exemplified by, but not limited to vinyl, allyl, butenyl, hexenyl, and octenyl. The silicone resin typically contains less than 9 mol % alkenyl groups attached to silicon relative to the sum of all silicon atoms in the alkenyl-functional MQ resin which totals 100 mol %, preferably, the alkenyl-functional MQ resin contains 1 to 7 mol % alkenyl groups, and more preferably 2 to 5 mol % alkenyl groups. The silicone resin typically has a weight-average molecular weight (M_{w}) of from 1,000 to 50,000 Daltons, alternatively from 2,000 to 30,000, alternatively 3,000 to 25,000 where the molecular weight is determined by gel permeation chromatography employing a light-scattering detector, a refractive index detector, and a viscosity detector and employing polystyrene standards. The silicone resin is typically delivered in a hydrocarbon or silicone solvent, free from solvent the silicone resin is typically a solid. Non-limiting examples of hydrocarbon solvents are aliphatic hydrocarbons, aromatic hydrocarbons, alcohols, esters, ethers, glycols, or ketones. Alternatively, the silicone resin could be delivered in a silicone solvent. Non-limiting examples of silicone solvents could be linear or cyclic polydimethylsiloxanes. The silicone resin typically contains less than 5% (w/w), alternatively less than 2% (w/w), alternatively less than 1.2% (w/w), of silicon-bonded hydroxy groups, as determined by NMR. The silicone resin can be a single silicone resin or a mixture comprising two or more different silicone resins, each as described above. Alternatively component (a) is a silicone resin having the formula: (R¹R²₂SiO_{1/2})_{w}(SiO_{4/2})_{z} wherein R¹ is vinyl, R² is methyl, w is from 0.3 to 0.6, z is from 0.4 to 0.7, and the value of w+z is 1.0.

Methods of preparing silicone resins are well known in the art. In some aspects, the resin is made by treating a resin copolymer produced by a silica hydrosol capping process with an alkenyl containing endblocking agent. This preferably includes reacting a silica hydrosol under acidic conditions with a hydrolysable triorganosilane such as trimethylchlorosilane, a siloxane such as hexamethyldisiloxane, and combinations thereof, and then recovering a copolymer having M (R₃SiO_{1/2}) units and Q (SiO_{4/2}) units including 2 to 5 wt % hydroxyl groups. The copolymer may be further reacted with an endblocking agent including unsaturated organic groups and an endblocking agent free of aliphatic unsaturation in amounts sufficient to provide 3 to 9 mol % of unsaturated organofunctional M or D units in the resin relative to the sum of all M, D (R₂SiO_{2/2}) units, and Q units comprising the resin. Suitable endblocking agents include silazanes, siloxanes, silanes, and combinations thereof.

With regard to component (b), the polyether, the value of c is from 0 to 50, alternatively from 0 to 24, or alternatively 0 to 14, and the value of d is form 0 to 50, alternatively from 0 to 20, or alternatively 0 to 7. The polyoxyalkylene useful as this component is a polyoxyalkylene that is terminated at each molecular chain end (i.e. alpha and omega positions) with an unsaturated aliphatic hydrocarbon group containing 2 to 12 carbon atoms. The polyoxyalkylene may result from the polymerization of ethylene oxide or propylene oxide. The polyoxyalkylene group may comprise oxyethylene units (C₂H₄O) or oxypropylene units (C₃H₆O). Typically, the polyoxyalkylene group comprises a majority of polyoxyethylene groups. In some embodiments a combination of two or more polyoxyalkylene groups can be advantageous. The unsaturated aliphatic hydrocarbon group can be an alkenyl or alkynyl group. Representative non-limiting examples of the alkenyl groups are: H₂C═CH—, H₂C═CH—CH₂—, H₂C═C(CH₃)CH₂—, H₂C═CHCH₂CH₂—, H₂C═CHCH₂CH₂CH₂₋, and H₂C═CHCH₂CH₂CH₂CH₂—. Representative, non-limiting examples of alkynyl groups are: HC≡C—, HC≡CCH₂—, HC≡CC(CH₃)H—, HC≡CC(CH₃)₂—, HC≡CC(CH₃)₂CH₂—. In one embodiment, the polyoxylalkylene is selected from H₂C═C(CH₃)CH₂O(C₂H₄O)_{c}CH₂(CH₃)C═CH₂ and H₂C═C(CH₃)CH₂O(C₃H₆O)_{d}CH₂(CH₃)C═CH₂ where c and d are defined as above.

Polyoxyalkylenes having an unsaturated aliphatic hydrocarbon group at each molecular terminal_{[BTD(1]} are known in the art, and many are commercially available. Polyoxyalkylenes having an unsaturated aliphatic hydrocarbon group at each molecular terminal_{[BTD(2]} are commercially available from NOF (Nippon Oil and Fat, Tokyo, Japan) and Clariant Corp. (Muttenz, Switzerland).

With regard to component (c), the Si—H functional organopolysiloxane, c is 0 or 1, R can be identical or different and is a monovalent, optionally substituted hydrocarbon with 1 to 18 carbon atoms per molecule, a and b are integers, with the proviso that the sum of a+b is 0 to 300, alternatively 4 to 200, alternatively 6 to 110, and that component (c) contains Si-bonded hydrogen in amounts from 0.01 to 0.4 wt %, preferably 0.02 to 0.2 wt %, more preferably 0.025 to 0.09 wt %. In a preferred embodiment, b is 0; c is 1; and a is from 6 to 110. R can be an alkyl group or aryl group optionally substituted with hyrdrocarbon groups such as methyl, ethyl, propyl, butyl, hexyl, pentyl, octyl, and decyl, or an aryl group such as phenyl. In one aspect, R is methyl. Component (c) can further comprise two or more SiH functional organopolysiloxanes which can be the same or different as desired. Alternatively (c) is an SiH functional organopolysiloxane having the general formula: HR₂SiO(R₂SiO)ₐ(RHSiO)_{b}SiR₂H wherein R is methyl and the sum of a+b is 4 to 200 or alternatively 6 to 110.

Component (d) the hydrosilylation catalyst can be any catalyst typically employed for hydrosilylation reactions. Typically the hydrosilylation catalyst is any platinum group metal-containing catalyst. By platinum group it is meant ruthenium, rhodium, palladium, osmium, iridium and platinum and complexes thereof. Platinum group metal-containing catalysts useful in preparing the compositions of the present invention are the platinum complexes prepared as described by Willing, U.S. Pat. No. 3,419,593, and Brown et al, U.S. Pat. No. 5,175,325. Other examples of useful platinum group metal-containing catalysts can be found in Lee et al., U.S. Pat. No. 3,989,668; Chang et al., U.S. Pat. No. 5,036,117; Ashby, U.S. Pat. No. 3,159,601; Lamoreaux, U.S. Pat. No. 3,220,972; Chalk et al., U.S. Pat. No. 3,296,291; Modic, U.S. Pat. No. 3,516,946; Karstedt, U.S. Pat. No. 3,814,730; and Chandra et al., U.S. Pat. No. 3,928,629. The platinum group-containing catalyst can be platinum group metal, platinum group metal deposited on a carrier such as silica gel or powdered charcoal, or a compound or complex of a platinum group metal. Preferred platinum-containing catalysts include chloroplatinic acid, either in hexahydrate form or anhydrous form, and or a platinum-containing catalyst which is obtained by a method comprising reacting chloroplatinic acid with an aliphatically unsaturated organosilicon compound such as divinyltetramethyldisiloxane, or alkene-platinum-silyl complexes as described in U.S. Pat. No. 6,605,734, such as (COD)Pt(SiMeCl₂)₂, where COD is 1,5-cyclooctadiene and Me is methyl. These alkene-platinum-silyl complexes may be prepared, for example by mixing 0.015 mole (COD)PtCl₂ with 0.045 mole COD and 0.0612 moles HMeSiCl₂. The appropriate amount of the catalyst will depend upon the particular catalyst used. The platinum catalyst should be present in an amount sufficient to provide at least 2 parts per million (ppm), alternatively 4 to 200 ppm of platinum based on total weight percent solids (all non-solvent ingredients) in the composition. Typically, the platinum is present in an amount sufficient to provide 4 to 150 weight ppm of platinum on the same basis. The catalyst may be added as a single species or as a mixture of two or more different species.

Generally, the carrier solvent, component (e), comprises at least one miscible fluid that is sufficiently compatible with the reaction mixture. In some aspects; suitable miscible fluids may be selected from silicones and organic compounds and mixtures thereof. Alternatively, the solvent could be a silicone polyether. Examples of suitable silicone carrier solvents include but are not limited to, low molecular weight linear or cyclic volatile silicones; non-volatile alkyl or aryl silicones; and low molecular weight linear or cyclic functional silicones. The silicone miscible fluid may be a single silicone or a mixture of silicones. In some aspects, the solvent is a low molecular weight volatile methyl silicone (VMS) having an average unit formula of (CH₃)ₓSiO_{(4-x)/2} in which x has an average value of from 2 to 3. Representative units in such VMS compounds are (CH₃)₃SiO_{1/2} units and (CH₃)₂SiO_{2/2} units, and there additionally may be CH₃SiO_{3/2} units and/or SiO_{4/2} units that result in the formulation of branched, linear or cyclic volatile methyl silicones. Linear VMS have the formula (CH₃)₃SiO{(CH₃)₂SiO}_{y}Si(CH₃)₃ where y is 0 to 5. Cyclic VMS have the formula {(CH₃)₂SiO}_{z} where z is 3 to 6. Typically these volatile methyl silicones have boiling points less than about 250° C. and viscosities from about 0.65 to 5.0 centistokes (mm²/s).

In some aspects, suitable silicone carrier solvents include, but are not limited to, linear volatile methyl silicones, such as hexamethyldisiloxane, octamethyltrisiloxane, decamethyltetrasiloxane, dodecamethylpentasiloxane, tetradecamethylhexasiloxane, hexadecamethylheptasiloxane, cyclic volatile methyl silicones, such as hexamethylcyclotrisiloxane, octamethylcyclotetrasiloxane, decamethylcyclopentasiloxane, and dodecamethylcyclohexasiloxane; and branch volatile methyl silicones, such as a heptamethyl-3-{(trimethylsilyl)oxy}trisiloxane, hexamethyl-3,3,bis-{(trimethylsilyl)oxy}trisiloxane, and pentamethyl {(trimethylsilyl)oxy}cyclotrisiloxane.

In some aspects, suitable non-volatile alkyl or arylsilicones that can be used as a carrier solvent include, but are not limited to, linear poly alkyl or aryl silicones; such as compounds of the formula R⁴₃SiO(R⁴₂SiO)ₘSiR⁴₃; and cyclic poly alkyl or aryl silicones, such as compounds of the formula (R⁴₂SiO)ₙ wherein R⁴ is an alkyl group of 1 to 6 carbon atoms, or an aryl group such as phenyl, m has a value of 0 to 80, preferably from 0 to 20 and n has a value of 0 to 9, preferably 4 to 6. These silicones have viscosities generally in the range of about 1 to 100 centistokes (mm²/s). Other representative low molecular weight non-volatile silicones have the general structure R⁵₃SiO(R⁵R⁶SiO)ₚSiR⁵₃ where p has a value to provide polymers with a viscosity in the range of about 100 to 10,000 centistokes (mm²/s) and R⁵ and R⁶ are alkyl radicals of 1 to 30 carbon atoms, or an aryl group such as phenyl. Typically, the value of p is about 60 to 600. Non-volatile polysiloxanes may be exemplified by, but are not limited to, polydimethylsiloxane, polydiethylsiloxane, polymethylethylsiloxane, polymethylphenylsiloxane, and polydiphenylsiloxane.

In some aspects, suitable organic carrier solvents include isododecane, isohexadecane, ethyl acetate, ethyl alcohol, isopropyl alcohol, ester palmitate, propylene glycol, C12-15 alkyl benzoate, caprylic/capric triglyceride, coco-caprylate/caprate, diisopropyl adipate, propyl propionate, isobutyl acetate, diisostearyl fumarate, diethyl carbonate, dicaprylyl carbonate, dicaprylyl ether, propylene carbonate, diisostearyl malate, isocetyl stearate, isopropyl isostearate, isopropyl laurate, isopropyl myristate, isopropyl palmitate, isopropyl stearate, isostearyl benzoate, myristyl ether acetate w/propylene glycol, myristyl lactate, octyldodecyl stearoyl stearate, octylpalmitate, octylstearate, tridecyl neopentanoate, triisocetyl citrate, lauryl alcohol, oleyl alcohol, glyceryl trioctanate, polyglyceryl-3 diisostearate, mineral oil, dipropylene glycol, glycol ether, propylene glycol methylether acetate, propylene glycol methylether, diethylhexyl carbonate, glycerin, castor oil, lanolin oil, sunflower oil, C11-12 isoparaffin, polydecene, organic fats, oils, fatty acids, fatty alcohols, their derivatives and combinations thereof may also be used.

Component (II) in the film forming composition of the invention is a silicone resin having the formula: (R⁴₃SiO_{1/2})ᵤ(SiO_{4/2})ᵥ wherein R⁴ is a C₁ to C₁₀ hydrocarbon group free of aliphatic unsaturation, u is from 0.3 to 0.6, v is from 0.4 to 0.7, and the value of u+v is 1.0. R⁴ can be an alkyl group or aryl group free of aliphatic unsaturation such as methyl, ethyl, propyl, propyl, butyl, hexyl, pentyl, octyl, and decyl, or an aryl group such as phenyl. In one aspect, R⁴ is methyl. The silicone resin of Component (II) typically has a weight-average molecular weight (M_{w}) of from 1,000 to 50,0000 Daltons, alternatively from 2,000 to 30,000, alternatively 3,000 to 25,000 where the molecular weight is determined by gel permeation chromatography employing a light-scattering detector, a refractive index detector, and a viscosity detector and employing polystyrene standards. The silicone resin of Component (II) is typically delivered in a hydrocarbon or silicone solvent, free from solvent the silicone resin is typically a solid. Non-limiting examples of hydrocarbon solvents are aliphatic hydrocarbons, aromatic hydrocarbons, alcohols, esters, ethers, glycols, or ketones. Alternatively, the silicone resin could be delivered in a silicone solvent. Non-limiting examples of silicone solvents could be linear or cyclic polydimethylsiloxanes. The silicone resin of (II) typically contains less than 5% (w/w), alternatively less than 4% (w/w), alternatively less than 3.5 wt % (w/w), of silicon-bonded hydroxy groups, as determined by NMR. The silicone resin of (II) can be a single silicone resin or a mixture comprising two or more different silicone resins, each as described above. Methods of preparing silicone resins are well known in the art. In some aspects, the resin is made by treating a resin copolymer produced by a silica hydrosol capping process with an alkyl containing endblocking agent. This preferably includes reacting a silica hydrosol under acidic conditions with a hydrolysable triorganosilane such as trimethylchlorosilane, a siloxane such as hexamethyldisiloxane, and combinations thereof, and then recovering a copolymer having M and Q groups including 2 to 5 wt % hydroxyl groups. The silicone resin of Component (II) can then be solvent exchanged into a carrier solvent or delivered as a flake or powder.

The hydrosilylation reaction product is the reaction product of components (a) through (e) above and is accomplished by mixing the ingredients together using any known methods in the art, including stirring them together, using equipment such as a mechanical stirrer, simple propeller mixers, Brookfield counter-rotating mixers, or homogenizing mixers. The ingredients can be mixed together at room temperature or can be heated, for example at a temperature of 50 to 110° C. In the reaction, one or more alkenyl functional silicone resins (component (a)) and one or more polyethers (component (b)) can be reacted with one or more SiH functional organopolysiloxanes (component (c)) in the presence of a hydrosilylation catalyst (component (d)) and carrier solvent (e) in a single reaction step. In various embodiments the alkenyl functional silicone resin (a) could be reacted with the SiH functional organopolysiloxane (c) with a molar excess of SiH. The remaining SiH groups could then be reacted with the alkenyl polyether. In another embodiment, the alkenyl polyether (b) could be reacted with the SiH functional organopolysiloxane (c) with a molar excess of SiH. The remaining SiH groups could then be reacted with the alkenyl polyether.

The hydrosilylation reaction product includes residual alkenyl or SiH functionality (e.g. as the result of the reaction between the alkenyl functional silicone resin (a), the alkenyl polyether (b), and the SiH functional organopolysiloxane (c). In various embodiments, the alkenyl or SiH functionality may be observed on a parts per million (ppm) or parts per billion (ppb) level, based on a total weight of the hydrosilylation reaction product. In other embodiments, the alkenyl or SiH functionality is understood based on a molar ratio of alkenyl to SiH functionality of the reactants (e.g. the alkenyl polyether, SiH functional organopolysiloxane, and alkenyl functional silicone resin). For example, the ratio_{[BTD(3]} of alkenyl to SiH units used to form the hydrosilylation product may be from 10:1 or 1:10. In various embodiments, this ratio can range from 8:1 to 1.5:1, or preferably from 6:1 to 2:1, and most preferably from 4:1 to 2:1. It is contemplated that any and all values or ranges of values between those described may also be utilized. The stoichiometry of the reaction should be tailored to prevent from forming a gelled product that is insoluble in a carrier solvent.

For the ingredients relative to themselves (excluding solvent as an ingredient) the hydrosilylation reaction product typically comprises: 1 to 90 wt %, alternatively 20 to 75 wt %, or alternatively 30 to 50 wt % of SiH functional organopolysiloxane (c), 1 to 90 wt %, alternatively 10-75 wt %, or alternatively 35 to 60 wt % alkenyl functional silicone resin (a), and 0.2 to 35 wt %, alternatively 2.0 to 25 wt %, or alternatively 5 to 15 wt % alkenyl polyether (b). In the total formulation including the solvent the amounts are 0.2 to 54 wt %, or alternatively 5 to 42 wt %, or alternatively 9 to 25 wt % SiH functional organopolysiloxane (c), 0.2 to 54 wt %, or alternatively 2.5 to 42 wt %, or alternatively 10.5 to 30 wt % alkenyl functional silicone resin (a), 0.04 to 21 wt %, alternatively 0.5-14 wt %, or alternatively 1.5 to 7.5 wt % alkenyl polyether (b), and 40 to 80 wt %, alternatively 45-75 wt %, or alternatively 50 to 70 wt % carrier solvent (e). In all embodiments, the hydrosilylation product should remain in solution and should not gel.

The components containing groups capable of reacting with SiH functionalities (e.g. alkenyl functional silicone resin (a) and the alkenyl polyether(b)) can be varied relative to one another to produce the desired ratio of total alkenyl groups to SiH functionality. For example the contribution of total moles of alkenyl groups could be normalized and divided between the alkenyl functional silicone resin and the alkenyl polyether. For example, the normalized moles of alkenyl groups on the alkenyl polyether could range relative to the normalized moles of alkenyl groups on the alkenyl functional silicone resin typically from 10:1 or 1:10.

An advantageous embodiment of the present invention is the post addition of a non-functional silicone resin (Component (II)) to the hydrosilylation product to improve rub-off resistance of a film of the product. In the preferred embodiment, the non-functional silicone resin (II) is added to the hydrosilylation reaction product in the carrier solvent to increase the overall MQ content of the solution. In applications where the combination of silicone resin (II) and a polyorganosiloxane is delivered in the presence of a carrier solvent, advantageous benefits are realized after the volatile carrier has evaporated from the product resulting in a film. The film is then represented solely as a combination of silicone resin (II) and a polyorganosiloxane. In these instances, the film can be described as having a silicone resin content relative to the total of all non-volatile components. In this invention, the alkenyl functional silicone resin (a) and the non-functional silicone resin (II) are considered to both contribute to the silicone resin content in the non-volatile components in which the non-volatile components can refer to the hydrosilylation product of the desired reaction.

In one embodiment, the non-functional silicone resin (II) is added to the hydrosilylation product (I) to achieve a total silicone resin content relative to the total of all non-volatile components of 45 to 90 wt % silicone resin (II), alternatively 55 to 80 wt % silicone resin (II); and alternatively 65 to 75 wt % silicone resin (II).

In a further embodiment, the non-functional silicone resin (II) is added prior to the hydrosilylation reaction which produces component (I). The non-functional silicone resin (II) could be added to the hydrosilylation reaction product (I) as a solution in the same solvent as that utilized for the hydrosilylation reaction product (I) or in a different solvent. Alternatively, the non-functional silicone resin (II) could be added to the hydrosilylation reaction product (I) as a solid free from a solvent either as a powder or as a flake.

The combination of the hydrosilylation reaction product (I) and non-functional silicone resin (II) can be delivered in the carrier solvents used for the hydrosilylation reaction. Alternatively, the combination of the hydrosilylation reaction product (I) and non-functional silicone resin (II) can be solvent exchanged such that the initial solvent is removed and the combination is delivered in a secondary carrier solvent or blend of solvents. In another embodiment, the combination of the hydrosilylation reaction product (I) and non-functional silicone resin (II) can be delivered as a solid flake or powder. In a further embodiment, the combination will be passed through a twin screw extruder to remove solvent and flaked.

The film forming composition of this invention is useful in manufacturing personal care compositions, which may also be described as a personal care product composition. The personal care composition includes the film forming composition described above. The personal care composition may be in the form of a cream, a gel, a powder, a paste, or a freely pourable liquid. Generally, such compositions can be prepared at room temperature if no solid materials at room temperature are present in the compositions, using simple propeller mixers, Brookfield counter-rotating mixers, or homogenizing mixers. No special equipment or processing conditions are typically required. Depending on the type of form made, the method of preparation will be different, but such methods are well known in the art.

The personal care composition may be functional with respect to the portion of the body to which it is applied, cosmetic, therapeutic, or some combination thereof. Conventional examples of such products include, but are not limited to, antiperspirants and deodorants, skin care creams, skin care lotions, moisturizers, facial treatments, such as acne or wrinkle removers, personal and facial cleansers, bath oils, perfumes, colognes, sachets, sunscreens, pre-shave and after-shave lotions, shaving soaps, and shaving lathers, hair shampoos, hair conditioners, hair colorants, hair relaxants, hair sprays, mousses, gels, permanents, depilatories, and cuticle coats, make-ups, color cosmetics, foundations, concealers, blushes, lipsticks, eyeliners, mascara, oil removers, color cosmetic removers, and powders, medicament creams, pastes or sprays including anti-acne, dental hygienic, antibiotic, healing promotive, nutritive and the like, which may be preventative and/or therapeutic. In general, the personal care composition may be formulated with a carrier that permits application in any conventional form, including but not limited to liquids, rinses, lotions, creams, pastes, gels, foams, mousses, ointments, sprays, aerosols, soaps, sticks, soft solids, solid gels, and gels. Suitable carriers are appreciated in the art.

### EXAMPLES

Formulation Materials:

Synthetic Ingredients:

SiH Polymer 1—an SiH organopolysiloxane polymer having the formula H(CH₃)₂SiO((CH₃)₂SiO)₁₀₀Si(CH₃)₂H, Calculated Mw=7668; Calculated wt % SiH=0.0261 wt %

Vinyl MQ Resin 1—an alkenyl functional MQ resin having the formula ((CH₃)₃SiO_{1/2})_{0.45}(CH₂═CH(CH₃)₂SiO_{1/2})_{0.04}(SiO_{4/2})_{0.51} containing: 1.2 wt % vinyl, 1.0 wt % OH-solvent exchanged into isododecane and delivered as a 60% solution, Mn=7010; Mw=20700

Solid MQ Resin 1—an MQ resin having the formula ((CH₃)₃SiO_{1/2})_{0.44}(SiO_{4/2})_{0.56} dissolved into isododecane at 70% solids

Solid MQ Resin 2—an MQ resin having the formula ((CH₃)₃SiO_{1/2})_{0.44}(SiO_{4/2})_{0.56} dissolved into ethyl acetate at 70% solids. Mn=3600; Mw=23000

Polyether 1 a bis-methallyl terminated poly(ethylene oxide) having the formula CH₂═C(CH₃)CH₂—O—(CH₂CH₂O)₁₄—CH₂(CH₃)C═CH₂

Polyether 2—a bis-methallyl terminated poly(propylene oxide) having the formula CH₂═C(CH₃)CH₂—O—(CH₂CH₂CH₂O)₇—CH₂(CH₃)C═CH₂

Platinum Catalyst—a 1.0 wt % solution in isopropanol of Pt (IV) complexes with 1,3-diethenyl-1,1,3,3-tetramethyldisiloxane

Isododecane 2,2,4,6,6-pentamethylheptane

Ethyl acetate

Isobutyl acetate

Propyl propionate

2-propanol

Cosmetic Ingredients:

Emulsifier Blend: Lauryl, PEG-10, and a tris(trimethylsilyl) modified polydimethylsiloxane (also known as an alkyl, polyethoxylated, silyl dendrimer-modifiedpolydimethylsiloxane) having the following formula:

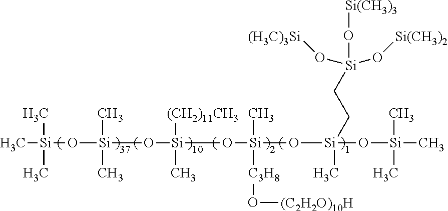

Sodium Chloride

Glycerin

Phenoxyethanol—Euxyl® PE 9010—Schulke & Mayr GmbH

Iron Oxide (C177499), Dimethicone—SA-C335000-10/Miyoshi Europe S.A.S.

Iron Oxide (C177491), Dimethicone—SA-C332199-10/Miyoshi Europe S.A.S.

Iron Oxide (C177492), Dimethicone—SA-C331700-10/Miyoshi Europe S.A.S.

Titanium Dioxide, Dimethicone—SA-TAO-77891/Miyoshi Europe S.A.S.

Caprylyl Methicone (also known as 3-octyl, heptamethyltrisiloxane) having the formula:

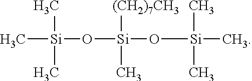

Benchmark Materials (Control):

Fluid 1—a 50% solution of an MQ resin having the formula ((CH₃)₃SiO_{1/2})_{0.44}(SiO_{4/2})_{0.56} in decamethylcyclopentasiloxane

Fluid 2—a 75% solution of silicone resin having the formula ((CH₃CH₂CH₂)SiO_{3/2})_{1.0} in isododecane

Acrylate Copolymer—a 40% solution of methacrylate/butyl acrylate co-polymer with a siloxane dendrimer in isododecane

### Example Method A

A 500 mL three-neck, round bottom flask equipped with a condenser and a nitrogen line, a glass stir shaft, a PTFE stir paddle, and a thermocouple were utilized in this reaction. For Example A1: to the flask, 38.35 grams of SiH Polymer 1, 53.24 grams Vinyl MQ Resin 1, 9.77 grams Polyether 1, and 58.76 grams of 2-propanol (solvent) were added. The contents of the flask were stirred at 250 rpm and heated to 70° C. Once the temperature reached or exceeded 70° C., the Platinum Catalyst Solution (containing 20 ppm Platinum relative to the entire formulation) was added. The mixture was allowed to react for 2 hours. At the conclusion of this time, heating was discontinued and the product collected. This same procedure was used to make Examples A2 through A11 with the amounts of the ingredients shown in Table 1 below.

TABLE 1

| | A1 | A2 | A3 | A4 | A5 | A6 |
|---|---|---|---|---|---|---|
| SiH Polymer 1 (grams) | 38.35 | 38.35 | 38.35 | 38.35 | 38.35 | 38.35 |
| Vinyl MQ Resin 1 | 53.24 | 76.05 | 114.08 | 15.21 | 38.03 | 76.05 |
| (grams) | | | | | | |
| Polyether 1 (grams) | 9.77 | 7.51 | 3.76 | — | — | — |
| Polyether 2 (grams) | — | — | — | 9.71 | 8.09 | 5.39 |
| Platinum Catalyst (ppm) | 20 | 20 | 20 | 20 | 20 | 20 |
| Solvent | 2- | 2- | 2- | isododecane | isododecane | isododecane |
| | propanol | propanol | propanol | | | |
| Solvent Weight (grams) | 58.76 | 61.07 | 64.92 | 51.10 | 54.05 | 58.95 |
| Total (grams) | 160.12 | 182.98 | 221.11 | 114.37 | 138.52 | 178.74 |
| wt % solids | 50.0% | 50.0% | 50.0% | 50.0% | 50.0% | 50.0% |
| Vi:SiH molar ratio | 4 | 4 | 4 | 4 | 4 | 4 |
| Vi Resin/allyl Polyether | 0.35 | 0.50 | 0.75 | 0.10 | 0.25 | 0.50 |
| Molar Ratio | | | | | | |
| wt % EO (solids basis) | 12.2% | 8.2% | 3.4% | — | — | — |
| wt % PO (solids basis) | — | — | — | 17.0% | 11.7% | 6.0% |

| | A7 | A8 | A9 | A10 | A11 |
|---|---|---|---|---|---|
| SiH Polymer 1 (grams) | 38.35 | 38.35 | 38.35 | 38.35 | 38.35 |
| Vinyl MQ Resin 1 | 114.08 | 76.05 | 76.05 | 76.05 | 38.03 |
| (grams) | | | | | |
| Polyether 1 (grams) | — | 1.35 | 2.7 | 4.05 | 4.05 |
| Polyether 2 (grams) | 2.7 | 5.63 | 3.76 | 1.88 | 5.63 |
| Platinum Catalyst (ppm) | 20 | 20 | 20 | 20 | 20 |
| Solvent | isododecane | isododecane | isododecane | isododecane | isododecane |
| Solvent Weight (grams) | 63.86 | 60.54 | 60.01 | 59.48 | 55.64 |
| Total (grams) | 218.99 | 181.92 | 180.87 | 179.81 | 141.7 |
| wt % solids | 50.0% | 50.0% | 50.0% | 50.0% | 50.0% |
| Vi:SiH molar ratio | 4 | 4 | 4 | 4 | 4 |
| Vi MQ Resin/Polyether | 0.75 | 0.50 | 0.50 | 0.50 | 0.25 |
| Molar Ratio | | | | | |
| wt % EO (solids basis) | — | 6.2% | 4.2% | 2.1% | 8.0% |
| wt % PO (solids basis) | 2.5% | 1.5% | 3.0% | 4.5% | 5.7% |

### Example Method B

A 1000 mL three-neck, round bottom flask equipped with a condenser and a nitrogen line, a glass stir shaft, a PTFE stir paddle, and a thermocouple were utilized in this reaction. To the flask, 280 grams of Solid MQ Resin 1 and 120 grams of isododecane were added. The contents of flask were heated to 35° C. and stirred until the solution was transparent and no remaining solid resin was observed. This method was used to produce MQ Resin Solution 1 (% solids=68.90%). This method was used to also produce MQ Resin Solution 2 (% solids=70.85%)

### Example Method C

MQ Resin Solution 1 was then combined with the reaction products from Formulations A1-A11 prepared according to Experimental Method A. The combined solutions were mixed on a Dental Mixer to produce a homogeneous solution. The resulting film forming compositions were labeled C1 C11 and are shown in Table 2 below.

TABLE 2

| | C1 | C2 | C3 | C4 | C5 | C6 |
|---|---|---|---|---|---|---|
| Experimental A Formulation | A1 | A2 | A3 | A4 | A5 | A6 |
| Experimental A Formulation wt | 19.32 | 21.16 | 20.73 | 22.22 | 21.65 | 21.27 |
| (grams) | | | | | | |
| MQ Resin Solution 1 wt (grams) | 17.57 | 9.26 | 3.69 | 24.77 | 16.91 | 8.74 |
| Resin content (solids basis) | 73% | 70% | 70% | 66% | 67% | 68% |

| | C7 | C8 | C9 | C10 | C11 |
|---|---|---|---|---|---|
| Experimental A Formulation | A7 | A8 | A9 | A10 | A11 |
| Experimental A Formulation wt | 20.65 | 21.59 | 21.52 | 21.28 | 21.9 |
| (grams) | | | | | |
| MQ Resin Solution 1 wt | 3.61 | 9.08 | 8.99 | 8.78 | 17.28 |
| Resin content (solids basis) | 71% | 71% | 70% | 69% | 69% |

### Foundation Formulation

The process to create the foundation was to mix phase A in a speed mixer at 2750 rpm for 1 minute. The amount of isododecane in the formulation was adjusted relative to the amount of solvent contained in the Film-Former (C1-C11) to bring the total of Phase A to 23 wt %. The water phase (phase B) was added in 4 equal parts with two minutes of mixing between each addition. A single phase should have resulted between each addition but if it did not, the solution was mixed longer before any further water was added. Phases A, B, and C are shown in Table 3 below. The end product formulation resulting from the mixing of Phases A, B, and C above with each of Formulations C1 through C11 above resulted in Formulations D1 through D11 which were then used as shown in Table 5 below.

TABLE 3
General Cosmetic Formulation

| | | Weight |
|---|---|---|
| | Ingredient Source | (grams) |
| Phase A | | |
| Emulsifier Blend | | 6 |
| Isododecane | — | Sum to |
| | | 23 wt. % |
| | | for phase A |
| Film-Former (C1 through C11) | — | 5% active |
| Phase B (Water Blend) | | |
| Water | Deionized lab water | 59.5 |
| Sodium Chloride | Fisher | 1 |
| Glycerin | Fisher | 5 |
| Phenoxyethanol, Ethylhexylglycerin | Euxyl ® PE | 1 |
| | 9010/Schülke & | |
| | Mayr GmbH | |
| Phase C (Pigment Blend) | | |
| Iron Oxide (CI77499), Dimethicone | SA-C335000-10/ | 0.07 |
| | Miyoshi Europe | |
| | S.A.S. | |
| Iron Oxide (CI77491), Dimethicone | SA-C332199-10/ | 0.25 |
| | Miyoshi Europe | |
| | S.A.S. | |
| Iron Oxide (CI77492), Dimethicone | SA-C331700-10/ | 1.09 |
| | Miyoshi Europe | |
| | S.A.S. | |
| Titanium Dioxide, Dimethicone | SA-TAO-77891/ | 5.81 |
| | Miyoshi Europe | |
| | S.A.S. | |
| Caprylyl Methicone | — | 3.28 |
| | Total | 100 |

TABLE 4
Masterbatches of Phase B and C in Preparation of Foundations

| | | | Weight |
|---|---|---|---|
| | Ingredient Source | wt. % | (grams) |
| Phase B (Water Blend) | | | |
| Water | | 59.5 | 1190 |
| Sodium Chloride | | 1 | 20 |
| Glycerin | | 5 | 100 |
| Phenoxyethanol, | Euxyl ® PE 9010/ | 1 | 20 |
| Ethylhexylglycerin | Schülke & | | |
| | Mayr GmbH | | |
| Phase C (Pigment Blend) | | | |
| Iron Oxide (CI77499), | SA-C335000-10/ | 0.07 | 1.75 |
| Dimethicone | Miyoshi Europe | | |
| | S.A.S. | | |
| Iron Oxide (CI77491), | SA-C332199-10/ | 0.25 | 6.25 |
| Dimethicone | Miyoshi Europe | | |
| | S.A.S. | | |
| Iron Oxide (CI77492), | SA-C331700-10/ | 1.09 | 27.25 |
| Dimethicone | Miyoshi Europe | | |
| | S.A.S. | | |
| Titanium Dioxide, | SA-TAO-77891/ | 5.81 | 145.25 |
| Dimethicone | Miyoshi Europe | | |
| | S.A.S. | | |
| Caprylyl Methicone | — | 3.28 | 82 |

TABLE 5
Formulations for D1-D11, Fluid 1, Fluid 2, and Acrylate Copolymer

| Formulation | | | | | | | | |
|---|---|---|---|---|---|---|---|---|
| Film-Former | Control | | | Acrylate | D1 | D2 | D3 | |
| Ingredient | Wt. % | Fluid 1 | Fluid 2 | Copolymer | C1 | C2 | C3 | |
| % Solids of Film- | | 50.0% | 75.0% | 40.0% | 59.3% | 55.0% | 52.3% | |
| Former | | | | | | | | |
| Phase A | | | | | | | | |
| Emulsifier Blend | 6 | 2.40 | 2.40 | 2.40 | 2.40 | 2.40 | 2.40 | |
| (grams) | | | | | | | | |
| lsododecane (grams) | make-up | 2.80 | 4.13 | 1.80 | 3.43 | 3.16 | 2.98 | |
| Film-Former | 5 | 4.00 | 2.67 | 5.00 | 3.37 | 3.64 | 3.82 | |
| (% active) | | | | | | | | |
| Phase B | | | | | | | | |
| Water Blend (grams) | 66.5 | 26.60 | 26.60 | 26.60 | 26.60 | 26.60 | 26.60 | |
| Phase C | | | | | | | | |
| Pigment Blend | 10.5 | 4.20 | 4.20 | 4.20 | 4.20 | 4.20 | 4.20 | |
| (grams) | | | | | | | | |
| (Iron Oxides/TiO2) | | | | | | | | |
| Total | | 40.00 | 40.00 | 40.00 | 40.00 | 40.00 | 40.00 | |
| Formulation | D4 | D5 | D6 | D7 | D8 | D9 | D10 | D11 |
| Ingredient | C4 | C5 | C6 | C7 | C8 | C9 | C10 | C11 |
| % Solids of | | | | | | | | |
| Film-Former | 60.9% | 58.9% | 55.7% | 52.3% | 53.4% | 54.3% | 55.0% | 57.1% |
| Phase A | | | | | | | | |
| Emulsifier Blend | 2.40 | 2.40 | 2.40 | 2.40 | 2.40 | 2.40 | 2.40 | 2.40 |
| (grams) | | | | | | | | |
| Isododecane | 3.51 | 3.40 | 3.21 | 2.97 | 3.06 | 3.12 | 3.16 | 3.29 |
| (grams) | | | | | | | | |
| Film-Former | 3.29 | 3.40 | 3.59 | 3.83 | 3.74 | 3.68 | 3.64 | 3.51 |
| (% active) | | | | | | | | |
| Phase B | | | | | | | | |
| Water Blend (grams) | 26.60 | 26.60 | 26.60 | 26.60 | 26.60 | 26.60 | 26.60 | 26.60 |
| Phase C | | | | | | | | |
| Pigment Blend | 4.20 | 4.20 | 4.20 | 4.20 | 4.20 | 4.20 | 4.20 | 4.20 |
| (grams) | | | | | | | | |
| (Iron Oxides/TiO2) | | | | | | | | |
| Total | 40.00 | 40.00 | 40.00 | 40.00 | 40.00 | 40.00 | 40.00 | 40.00 |

### Testing:

### Abrasion Testing

Abrasion resistance was measured on a water-in-oil foundation that contained the film formers.

The foundation was coated out using a 2 micron film gauge onto hydrated collagen (˜150 micron thickness) which had been pulled over and taped to a polycarbonate block. The foundation was allowed to dry overnight in a fume hood. Using a Gardner abrasion tester fitted with an adhesive strip of the loop side of Velcro, each sample was insulted for either 50 or 20 cycles, depending on whether the film was dry or wetted with sebum. Three blocks were tested per film-forming material. Each test material was tested dry. In subsequent testing several film-formers were tested for sebum resistance which requires adding a drop of artificial sebum to the coated block and evenly spreading it out and then waiting one minute before beginning the abrasion testing.

A Hunterlab colorimeter was used to measure the LAB (color) value for each block using a 0.5″ diameter filter. Measurements were taken after 0, 4, 8, 12, 16, 20, 30, 40, and 50 insults. If using sebum the testing stopped at 20 insults. From this a ΔΕ (or color change) was calculated to determine the resistance to abrasion.

### Film Flexibility and Shine

The coater and latex rubber used in this study are further mentioned below. A shine box, which is a wooden box with a light source, was utilized in order to visually measure shine. Coated rubber specimens were hung in the shine box in order to generate a clearer, more reproducible shine band for each test material.

Elastic Band—Latex Rubber:

0.33 mm thick specimens

Supplier: Four D Rubber Co. Ltd. in United Kingdom

Red latex rubber: to be used for translucent samples

White latex rubber: to be used for colored formulations

Coater:

Procedure:

1. The test material was coated onto the elastic band using a cube coater (Sheen brand ref# 1103A cube).

The substrate was affixed to a rigid and plane support (e.g. aluminum plate or table) and fastened with adhesive tape.

The coating had a length of 10 cm and a width of ⅔ cm.

The coating was drawn down on the shiny side of the elastic band.

For diluted formulations, the coating thickness was 50 μm and for the fully formulated foundation the thickness was 25 μm.

2. The films were dried overnight.

3. Once the film was dry, it was affixed to the substrate on a plane support and a picture was taken before any force was applied to the coating. Film characteristics were noted before elongation: presence of cracks, shine and/or whitening effect, tackiness and film homogeneity.

4. Then a ruler was placed above the substrate to monitor the percentage of elongation. An elongation of 200% was performed.

5. After performing the elongation step, a new picture was taken. Film characteristics were noted before and after elongation; specifically, the presence of cracks, shine and/or whitening effect, tackiness and film homogeneity.

### Contact Angle

Substrate:

Glass microscope slides were used as the substrate to determine water and sebum repellency of polymer coatings.

Coater:

Thin films were prepared using a cube applicator as the one supplied by Sheen Instruments. Quadruple, square or Mayer rod applicators can also be used for larger surfaces. 50 μm wet films were used when assessing film-forming properties of neat polymers (dispersed at 20 wt % in a volatile carrier) and 25 μm wet films were used when assessing the film-forming properties of finished cosmetic formulations.

Artificial Sebum Solution:

An artificial sebum with the following composition was prepared and used for sebum resistance testing:

TABLE 5

| Trade name | INCI name | Wt. % |
|---|---|---|
| glyceryl trioleate (65%, sigma) | Triolein | 60 |
| Oleic acid (grade 90% Aldrich) | Oleic acid | 20 |
| Pripure 3759 (Croda) | Squalane | 20 |

Ultrapure water (to be freshly prepared on the test day)

TABLE 6
Performance Summary for Neat and Fully-Formulated Film-Formers

| | | | | | | | Foundation |
|---|---|---|---|---|---|---|---|
| | Contact Angle | | Neat Film | | | Film-Forming | Abrasion (50 |
| Material | Water | Sebum | Tack | Shine | Flexibility | Formulation | Insults) |
| C1 | 107.1° | 77.6° | 3 | 4 | 4 | D1 | 4 |
| C2 | 108.4° | 78.0° | 4 | 4 | 4 | D2 | 4 |
| C3 | 104.4° | 46.7° | 4 | 4 | 4 | D3 | 4 |
| C4 | 105.5° | 28.8° | 4 | 2 | 4 | D4 | 2 |
| C5 | 108.6° | 38.0° | 4 | 3 | 4 | D5 | 3 |
| C6 | 106.0° | 46.4° | 4 | 3 | 4 | D6 | 2 |
| C7 | 104.5° | 46.0° | 4 | 2 | 4 | D7 | 3 |
| C8 | 106.5° | 66.7° | 4 | 3 | 4 | D8 | 4 |
| C9 | 105.5° | 52.1° | 3 | 1 | 4 | D9 | 2 |
| C10 | 103.3° | 49.1° | 3 | 3 | 4 | D10 | 1 |
| C11 | 107.0° | 68.9° | 3 | 3 | 4 | D11 | 2 |
| Fluid 1 | 95.9° | 33.9° | 4 | 2 | 1 | Fluid 1 | 3 |
| Fluid 2 | 97.1° | 36.0° | 2 | 3 | 3 | Fluid 2 | 3 |
| Acrylate | 99.0° | 49.1° | 4 | 2 | 1 | Acrylate | 4 |
| Copolymer | | | | | | Copolymer | |

Key for above table:
Tack (4 = no tack; 1 = very tacky)
Shine (4 = glossy; 1 = matte)
Flexibility after stretching (4 = highly flexible with no cracks; 1 = inflexible, cracked upon stretching)
Abrasion (4 = minimal reduction in color, 1 = substantial reduction in color)

1. A film forming composition that can produce a tack free surface comprising:
(I) a hydrosilylation reaction product of:
(a) a silicone resin having the formula: (R¹R²₂SiO_{1/2})_{w}(R¹R²SiO_{2/2})ₓ(R²SiO_{3/2})_{y}(SiO_{4/2})_{z} wherein R² is a C₁ to C₁₀ hydrocarbon group free of aliphatic unsaturation, R¹ is R² or an alkenyl group, w is from 0.3 to 0.6, x is from 0.0 to 0.2, y is 0, z is from 0.4 to 0.7, w+x+y+z=1.0, and w+z is from 0.85 to 1.0, with the proviso that the silicone resin (a) has at least two silicon-bonded alkenyl groups per number average molecule;
(b) a polyether compound having the formula: R³O(C₂H₄O)_{c}(C₃H₆O)_{d}R³ wherein R³ is a monovalent unsaturated aliphatic hydrocarbon group containing 2 to 12 carbon atoms, c is from 0 to 50, d is from 0 to 50, and the value of c+d is greater than zero;
(c) an SiH functional organopolysiloxane having the general formula: H_{c}R_{3-c}SiO(R₂SiO)ₐ(RHSiO)_{b}SiR_{3-c}H_{c} wherein c is 0 or 1, R can be identical or different and is a monovalent, optionally substituted hydrocarbon group with 1 to 18 carbon atoms per molecule, a and b are integers, with the proviso that the sum of a+b is 0 to 300, and wherein the organopolysilane contains SiH in amounts from 0.01 to 0.4 wt %;
(d) a hydrosilylation catalyst; and
(e) a carrier solvent; and
(II) a silicone resin having the formula: (R⁴₃SiO_{1/2})ᵤ(SiO_{4/2})ᵥ wherein R⁴ is a C₁ to C₁₀ hydrocarbon group free of aliphatic unsaturation, u is from 0.3 to 0.6, v is from 0.4 to 0.7, and the value of u+v is 1.0.

2. A composition according to claim 1, wherein (a) is a silicone resin having the formula: (R¹R²₂SiO_{1/2})_{w}(SiO_{4/2})_{z} wherein R¹ is vinyl, R² is methyl, w is from 0.3 to 0.6, z is from 0.4 to 0.7, and the value of w+z is 1.0.

3. A composition according to claim 2, wherein (b) is selected from H₂C═C(CH₃)CH₂O(C₂H₄O)_{c}CH₂(CH₃)C═CH₂ or H₂C═C(CH₃)CH₂O(C₃H₆O)_{d}CH₂(CH₃)C═CH₂ wherein c is from 0 to 14 and d is from 0 to 7.

4. A composition according to claim 3, wherein (c) is an SiH functional organopolysiloxane having the general formula: HR₂SiO(R₂SiO)ₐ(RHSiO)_{b}SiR₂H wherein the sum of a+b is 6 to 110 and R is methyl.

5. A composition according to claim 4, wherein (d) is a platinum group metal-containing catalyst.

6. A composition according to claim 5, wherein (e) is selected from linear volatile methyl silicones selected from hexamethyldisiloxane, octamethyltrisiloxane, decamethyltetrasiloxane, dodecamethylpentasiloxane, tetradecamethylhexasiloxane, or hexadecamethylheptasiloxane, cyclic volatile methyl silicones selected from hexamethylcyclotrisiloxane, octamethylcyclotetrasiloxane, decamethylcyclopentasiloxane, or dodecamethylcyclohexasiloxane; or branch volatile methyl silicones selected from heptamethyl-3-{(trimethylsilyl)oxy}trisiloxane, hexamethyl-3,3,bis-{(trimethylsilyl)oxytrisiloxane, or pentamethyl {(trimethylsilyl)oxy}cyclotrisiloxane, non-volatile polysiloxanes selected from polydimethylsiloxane, polydiethylsiloxane, polymethylethylsiloxane, polymethylphenylsiloxane, or, polydiphenylsiloxane, or organic carrier solvents selected from isododecane,isohexadecane, ethyl acetate, ethyl alcohol, isopropyl alcohol, ester palmitate, propylene glycol, C12-15 alkyl benzoate, caprylic/capric triglyceride, coco-caprylate/caprate, diisopropyl adipate, propyl propionate, isobutyl acetate, diisostearyl fumarate, diethyl carbonate, dicaprylyl carbonate, dicaprylyl ether, propylene carbonate, diisostearyl malate, isocetyl stearate, isopropyl isostearate, isopropyl laurate, isopropyl myristate, isopropyl palmitate, isopropyl stearate, isostearyl benzoate, myristyl ether acetate w/propylene glycol, myristyl lactate, octyldodecyl stearoyl stearate, octylpalmitate, octylstearate, tridecyl neopentanoate, triisocetyl citrate, lauryl alcohol, oleyl alcohol, glyceryl trioctanate, polyglyceryl-3 diisostearate, mineral oil, dipropylene glycol, glycol ether, propylene glycol methylether acetate, propylene glycol methylether, diethylhexyl carbonate, glycerin, castor oil, lanolin oil, sunflower oil, C11-12 isoparaffin, polydecene, organic fats, oils, fatty acids, fatty alcohols, or combinations thereof.

7. A composition according to claim 1, wherein R⁴ is methyl.

8. A composition according to claim 6, wherein R⁴ is methyl.

9. A composition according to claim 1, wherein:
(a) is a silicone resin having the formula: (R¹R²₂SiO_{1/2})_{w}(SiO_{4/2})_{z} wherein R¹ is vinyl, R² is vinyl, w is from 0.3 to 0.6, z is from 0.4 to 0.7, and the value of w+z is 1.0;
(b) is selected from H₂C═C(CH₃)CH₂O(C₂H₄O)_{c}CH₂(CH₃)C═CH₂ or H₂C═C(CH₃)CH₂O(C₃H₆O)_{d}CH₂(CH₃)C═CH₂ wherein c is from 0 to 14 and d is from 0 to 7;
(c) is an SiH functional organopolysiloxane having the general formula: HR₂SiO(R₂SiO)ₐ(RHSiO)_{b}SiR₂H wherein the sum of a+b is 6 to 110 and R is methyl;
(d) is a platinum group metal-containing catalyst;
(e) is selected from linear volatile methyl silicones selected from hexamethyldisiloxane, octamethyltrisiloxane, decamethyltetrasiloxane, dodecamethylpentasiloxane, tetradecamethylhexasiloxane, or hexadecamethylheptasiloxane, cyclic volatile methyl silicones selected from hexamethylcyclotrisiloxane, octamethylcyclotetrasiloxane, decamethylcyclopentasiloxane, or dodecamethylcyclohexasiloxane; or branch volatile methyl silicones selected from heptamethyl-3-{(trimethylsilyl)oxy}trisiloxane, hexamethyl-3,3,bis-{(trimethylsilyl)oxytrisiloxane, or pentamethyl {(trimethylsilyl)oxy}cyclotrisiloxane, non-volatile polysiloxanes selected from polydim ethylsiloxane, polydiethylsiloxane, polymethylethylsiloxane, polymethylphenylsiloxane, or, polydiphenylsiloxane, or organic carrier solvents selected from isododecane,isohexadecane, ethyl acetate, ethyl alcohol, isopropyl alcohol, ester palmitate, propylene glycol, C12-15 alkyl benzoate, caprylic/capric triglyceride, coco-caprylate/caprate, diisopropyl adipate, propyl propionate, isobutyl acetate, diisostearyl fumarate, diethyl carbonate, dicaprylyl carbonate, dicaprylyl ether, propylene carbonate, diisostearyl malate, isocetyl stearate, isopropyl isostearate, isopropyl laurate, isopropyl myristate, isopropyl palmitate, isopropyl stearate, isostearyl benzoate, myristyl ether acetate w/propylene glycol, myristyl lactate, octyldodecyl stearoyl stearate, octylpalmitate, octylstearate, tridecyl neopentanoate, triisocetyl citrate, lauryl alcohol, oleyl alcohol, glyceryl trioctanate, polyglyceryl-3 diisostearate, mineral oil, dipropylene glycol, glycol ether, propylene glycol methylether acetate, propylene glycol methylether, diethylhexyl carbonate, glycerin, castor oil, lanolin oil, sunflower oil, C11-12 isoparaffin, polydecene, organic fats, oils, fatty acids, fatty alcohols, or combinations thereof; and
(II) is a silicone resin having the formula: (R⁴₃SiO_{1/2})ᵤ(SiO_{4/2})ᵥ wherein R⁴ is methyl, u is from 0.3 to 0.6, v is from 0.4 to 0.7, and the value of u+v is 1.0.

10. A composition according to claim 1, wherein the hydrosilylation reaction product is produced by a method comprising mixing and optionally heating (a), (b), (c), (d), and (e).

11. A composition according to claim 9, wherein the hydrosilylation reaction product is produced by a method comprising mixing and optionally heating (a), (b), (c), (d), and (e).

12. A composition according to claim 10, wherein heating occurs at a temperature of 50 to 110° C.

13. A composition according to claim 11, wherein heating occurs at a temperature of 50 to 110° C.

14. A composition according to claim 1, wherein silicone resin (II) is added to the hydrosilylation product (I) to achieve a total MQ resin content relative to the total of all non-volatile components of 45 to 90 wt % silicone resin (II), alternatively 55 to 80 wt % silicone resin (II); and alternatively 65 to 75 wt % silicone resin (II).

15. A composition according to claim 9, wherein silicone resin (II) is added to the hydrosilylation product (I) to achieve a total MQ resin content relative to the total of all non-volatile components of 45 to 90 wt % silicone resin (II), alternatively 55 to 80 wt % silicone resin (II); and alternatively 65 to 75 wt % silicone resin (II).
